(19) United States
(12) Patent Application Publication
(10) Pub. No.: US 2025/0285161 A1
(43) Pub. Date: Sep. 11, 2025

(54) PERSONAL ASSISTANT ASSOCIATED WITH A MOBILE DEVICE OPERATING ON A 5G WIRELESS TELECOMMUNICATION NETWORK TO AID IN TRIP PLANNING

(71) Applicant: T-Mobile USA, Inc., Bellevue, WA (US)

(72) Inventor: Phi Hoang Nguyen, Lacey, WA (US)

(21) Appl. No.: 18/597,653

(22) Filed: Mar. 6, 2024

(51) Int. Cl. G06Q 30/0601; G06Q 10/109; G16H 20/10

(52) U.S. Cl. CPC G06Q 30/0631; G06Q 10/109; G16H 20/10

(57) ABSTRACT

The system obtains an indication of an activity, a start indicator associated with the activity, an end indicator associated with the activity, a destination associated with the activity, and a weather forecast associated with the destination between the start indicator and the end indicator. Based on the weather forecast and the activity, the system can determine, by the personal assistant, multiple items associated with the activity. The system determines whether a user is in possession of an item among the multiple items. Upon determining that the user is not in possession of the item, the system provides an instruction to obtain the item; otherwise, the system scans. Based on the scan, the system determines whether the item is in a satisfactory condition, and upon determining that the item is not in the satisfactory condition, the system provides the instruction to obtain the item.

### BACKGROUND

The proliferation of mobile apps appearing on customers' devices is overwhelming consumers. It seems there is an app for every conceivable purpose, with most being individual apps that either share some common functionalities or uniquely cater to specific needs. Due to privacy and/or security concerns, these apps typically do not share data with others unless they belong to the same family of company apps. The lack of data sharing between apps highlights the shortcomings of a fully integrated personal device capable of comprehensively understanding individual profiles, preferences, current and upcoming activities, personal health conditions, and similar information within family circles. This integration could enable the automatic recommendation of personalized content and the resolution of needs in a timely manner. Without such integration, we continue to rely on human memory to remember and manually connect and correlate information around us.

### BRIEF DESCRIPTION OF THE DRAWINGS

Detailed descriptions of implementations of the present invention will be described and explained through the use of the accompanying drawings.

FIG. 1 is a block diagram that illustrates a wireless telecommunication network in which aspects of the disclosed technology are incorporated.

FIG. 2 is a block diagram that illustrates an architecture including 5G core network functions (NFs) that can implement aspects of the present technology.

FIG. 3 is an overview of a personal digital assistant system to aid in planning a trip.

FIG. 4 shows the key components of a personal digital assistant system.

FIG. 5 shows a subsystem that recognizes a user's relationships and incorporates the relationships' preferences.

FIG. 6 presents a detailed flow of steps involved in recognizing the appropriate gear for an activity.

FIG. 7 illustrates monitoring of various aspects related to the destination and actions to take if changes occur.

FIG. 8 is a flowchart of a method to aid in trip planning.

FIG. 9 is a block diagram that illustrates an example of a computer system in which at least some operations described herein can be implemented.

The technologies described herein will become more apparent to those skilled in the art from studying the Detailed Description in conjunction with the drawings. Embodiments or implementations describing aspects of the invention are illustrated by way of example, and the same references can indicate similar elements. While the drawings depict various implementations for the purpose of illustration, those skilled in the art will recognize that alternative implementations can be employed without departing from the principles of the present technologies. Accordingly, while specific implementations are shown in the drawings, the technology is amenable to various modifications.

### DETAILED DESCRIPTION

Disclosed here is a system and method to aid in trip planning using a personal assistant associated with a mobile device operating on a 5G wireless telecommunication network. The system obtains an indication of an upcoming trip, a start date and time associated with the upcoming trip, an end date and time associated with the upcoming trip, a destination associated with the upcoming trip, and an activity associated with the destination. The system can obtain the information about the upcoming trip from a calendar or from emails and messages on the mobile device.

The system obtains a weather forecast associated with the destination between the start indicator and the end indicator. Based on the weather forecast and the activity associated with the destination, the system determines multiple items associated with the upcoming trip, such as clothing, equipment, legal documents, etc. The system determines whether a user is in possession of an item among the multiple items, and upon determining that the user is not in possession of the item among the multiple items, the system provides a link to purchase the item.

Upon determining that the user is in possession of the item among the multiple items, the system scans the item by, for example, using a camera or rangefinder of the mobile device. Based on the scan, the system can determine whether the item is in a satisfactory condition, such as whether the treads on the tires or the shoes are sufficiently deep, whether the clothes are not torn, whether the equipment is not damaged or broken, etc. Upon determining that the item is not in the satisfactory condition, the system provides the link to purchase the item. In addition, the system can create a packing list of the needed items.

The description and associated drawings are illustrative examples and are not to be construed as limiting. This disclosure provides certain details for a thorough understanding and enabling description of these examples. One skilled in the relevant technology will understand, however, that the invention can be practiced without many of these details. Likewise, one skilled in the relevant technology will understand that the invention can include well-known structures or features that are not shown or described in detail to avoid unnecessarily obscuring the descriptions of examples.

### Wireless Communications System

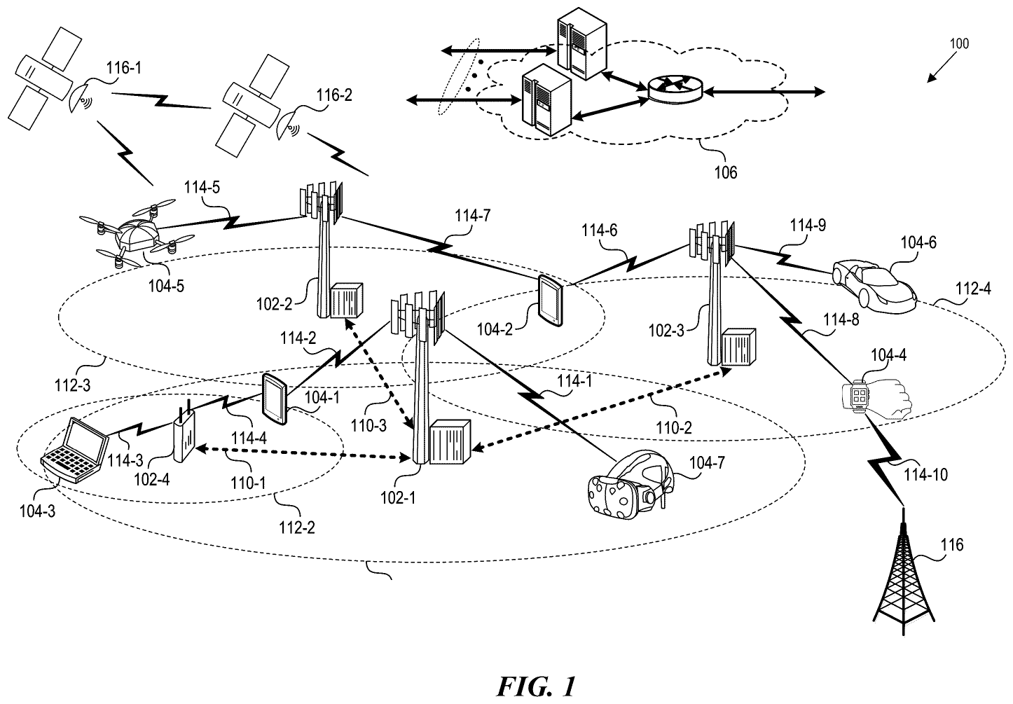

FIG. 1 is a block diagram that illustrates a wireless telecommunication network 100 (“network 100”) in which aspects of the disclosed technology are incorporated. The network 100 includes base stations 102-1 through 102-4 (also referred to individually as “base station 102” or collectively as “base stations 102”). A base station is a type of network access node (NAN) that can also be referred to as a cell site, a base transceiver station, or a radio base station. The network 100 can include any combination of NANs including an access point, radio transceiver, gNodeB (gNB), NodeB, eNodeB (eNB), Home NodeB or Home eNodeB, or the like. In addition to being a wireless wide area network (WWAN) base station, a NAN can be a wireless local area network (WLAN) access point, such as an Institute of Electrical and Electronics Engineers (IEEE) 802.11 access point.

The NANs of a network 100 formed by the network 100 also include wireless devices 104-1 through 104-7 (referred to individually as “wireless device 104” or collectively as “wireless devices 104”) and a core network 106. The wireless devices 104 can correspond to or include network 100 entities capable of communication using various connectivity standards. For example, a 5G communication channel can use millimeter wave (mmW) access frequencies of 28 GHz or more. In some implementations, the wireless device 104 can operatively couple to a base station 102 over a long-term evolution/long-term evolution-advanced (LTE/LTE-A) communication channel, which is referred to as a 4G communication channel.

The core network 106 provides, manages, and controls security services, user authentication, access authorization, tracking, Internet protocol (IP) connectivity, and other access, routing, or mobility functions. The base stations 102 interface with the core network 106 through a first set of backhaul links (e.g., S1 interfaces) and can perform radio configuration and scheduling for communication with the wireless devices 104 or can operate under the control of a base station controller (not shown). In some examples, the base stations 102 can communicate with each other, either directly or indirectly (e.g., through the core network 106), over a second set of backhaul links 110-1 through 110-3 (e.g., X1 interfaces), which can be wired or wireless communication links.

The base stations 102 can wirelessly communicate with the wireless devices 104 via one or more base station antennas. The cell sites can provide communication coverage for geographic coverage areas 112-1 through 112-4 (also referred to individually as “coverage area 112” or collectively as “coverage areas 112”). The coverage area 112 for a base station 102 can be divided into sectors making up only a portion of the coverage area (not shown). The network 100 can include base stations of different types (e.g., macro and/or small cell base stations). In some implementations, there can be overlapping coverage areas 112 for different service environments (e.g., Internet of Things (IoT), mobile broadband (MBB), vehicle-to-everything (V2X), machine-to-machine (M2M), machine-to-everything (M2X), ultra-reliable low-latency communication (URLLC), machine-type communication (MTC), etc.).

The network 100 can include a 5G network 100 and/or an LTE/LTE-A or other network. In an LTE/LTE-A network, the term “eNBs” is used to describe the base stations 102, and in 5G new radio (NR) networks, the term “gNBs” is used to describe the base stations 102 that can include mmW communications. The network 100 can thus form a heterogeneous network 100 in which different types of base stations provide coverage for various geographic regions. For example, each base station 102 can provide communication coverage for a macro cell, a small cell, and/or other types of cells. As used herein, the term “cell” can relate to a base station, a carrier or component carrier associated with the base station, or a coverage area (e.g., sector) of a carrier or base station, depending on context.

A macro cell generally covers a relatively large geographic area (e.g., several kilometers in radius) and can allow access by wireless devices that have service subscriptions with a wireless network 100 service provider. As indicated earlier, a small cell is a lower-powered base station, as compared to a macro cell, and can operate in the same or different (e.g., licensed, unlicensed) frequency bands as macro cells. Examples of small cells include pico cells, femto cells, and micro cells. In general, a pico cell can cover a relatively smaller geographic area and can allow unrestricted access by wireless devices that have service subscriptions with the network 100 provider. A femto cell covers a relatively smaller geographic area (e.g., a home) and can provide restricted access by wireless devices having an association with the femto unit (e.g., wireless devices in a closed subscriber group (CSG), wireless devices for users in the home). A base station can support one or multiple (e.g., two, three, four, and the like) cells (e.g., component carriers). All fixed transceivers noted herein that can provide access to the network 100 are NANs, including small cells.

The communication networks that accommodate various disclosed examples can be packet-based networks that operate according to a layered protocol stack. In the user plane, communications at the bearer or Packet Data Convergence Protocol (PDCP) layer can be IP-based. A Radio Link Control (RLC) layer then performs packet segmentation and reassembly to communicate over logical channels. A Medium Access Control (MAC) layer can perform priority handling and multiplexing of logical channels into transport channels. The MAC layer can also use Hybrid ARQ (HARQ) to provide retransmission at the MAC layer, to improve link efficiency. In the control plane, the Radio Resource Control (RRC) protocol layer provides establishment, configuration, and maintenance of an RRC connection between a wireless device 104 and the base stations 102 or core network 106 supporting radio bearers for the user plane data. At the Physical (PHY) layer, the transport channels are mapped to physical channels.

Wireless devices can be integrated with or embedded in other devices. As illustrated, the wireless devices 104 are distributed throughout the network 100, where each wireless device 104 can be stationary or mobile. For example, wireless devices can include handheld mobile devices 104-1 and 104-2 (e.g., smartphones, portable hotspots, tablets, etc.); laptops 104-3; wearables 104-4; drones 104-5; vehicles with wireless connectivity 104-6; head-mounted displays with wireless augmented reality/virtual reality (AR/VR) connectivity 104-7; portable gaming consoles; wireless routers, gateways, modems, and other fixed-wireless access devices; wirelessly connected sensors that provide data to a remote server over a network; IoT devices such as wirelessly connected smart home appliances; etc.

A wireless device (e.g., wireless devices 104) can be referred to as a user equipment (UE), a customer premises equipment (CPE), a mobile station, a subscriber station, a mobile unit, a subscriber unit, a wireless unit, a remote unit, a handheld mobile device, a remote device, a mobile subscriber station, a terminal equipment, an access terminal, a mobile terminal, a wireless terminal, a remote terminal, a handset, a mobile client, a client, or the like.

A wireless device can communicate with various types of base stations and network 100 equipment at the edge of a network 100 including macro eNBs/gNBs, small cell eNBs/gNBs, relay base stations, and the like. A wireless device can also communicate with other wireless devices either within or outside the same coverage area of a base station via device-to-device (D2D) communications.

The communication links 114-1 through 114-9 (also referred to individually as “communication link 114” or collectively as “communication links 114”) shown in network 100 include uplink (UL) transmissions from a wireless device 104 to a base station 102 and/or downlink (DL) transmissions from a base station 102 to a wireless device 104. The downlink transmissions can also be called forward link transmissions while the uplink transmissions can also be called reverse link transmissions. Each communication link 114 includes one or more carriers, where each carrier can be a signal composed of multiple sub-carriers (e.g., waveform signals of different frequencies) modulated according to the various radio technologies. Each modulated signal can be sent on a different sub-carrier and carry control information (e.g., reference signals, control channels), overhead information, user data, etc. The communication links 114 can transmit bidirectional communications using frequency division duplex (FDD) (e.g., using paired spectrum resources) or time division duplex (TDD) operation (e.g., using unpaired spectrum resources). In some implementations, the communication links 114 include LTE and/or mmW communication links.

In some implementations of the network 100, the base stations 102 and/or the wireless devices 104 include multiple antennas for employing antenna diversity schemes to improve communication quality and reliability between base stations 102 and wireless devices 104. Additionally or alternatively, the base stations 102 and/or the wireless devices 104 can employ multiple-input, multiple-output (MIMO) techniques that can take advantage of multi-path environments to transmit multiple spatial layers carrying the same or different coded data.

In some examples, the network 100 implements 6G technologies including increased densification or diversification of network nodes. The network 100 can enable terrestrial and non-terrestrial transmissions. In this context, a Non-Terrestrial Network (NTN) is enabled by one or more satellites, such as satellites 116-1 and 116-2, to deliver services anywhere and anytime and provide coverage in areas that are unreachable by any conventional Terrestrial Network (TN). A 6G implementation of the network 100 can support terahertz (THz) communications. This can support wireless applications that demand ultrahigh quality of service (QOS) requirements and multi-terabits-per-second data transmission in the era of 6G and beyond, such as terabit-per-second backhaul systems, ultra-high-definition content streaming among mobile devices, AR/VR, and wireless high-bandwidth secure communications. In another example of 6G, the network 100 can implement a converged Radio Access Network (RAN) and Core architecture to achieve Control and User Plane Separation (CUPS) and achieve extremely low user plane latency. In yet another example of 6G, the network 100 can implement a converged Wi-Fi and Core architecture to increase and improve indoor coverage.

### 5G Core Network Functions

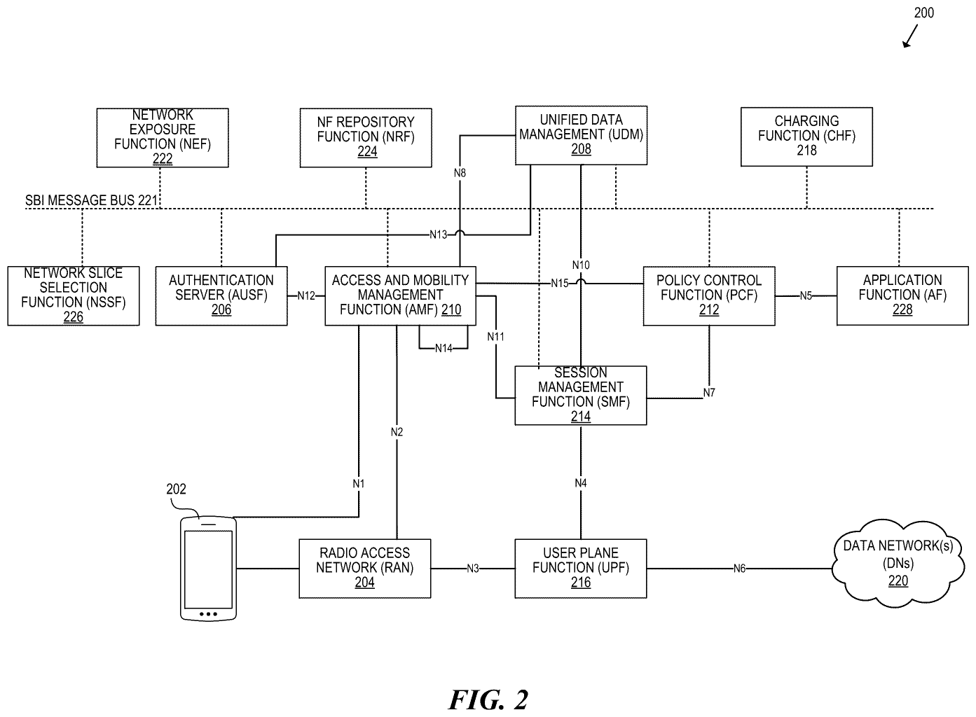

FIG. 2 is a block diagram that illustrates an architecture 200 including 5G core network functions (NFs) that can implement aspects of the present technology. A wireless device 202 can access the 5G network through a NAN (e.g., gNB) of a RAN 204. The NFs include an Authentication Server Function (AUSF) 206, a Unified Data Management (UDM) 208, an Access and Mobility management Function (AMF) 210, a Policy Control Function (PCF) 212, a Session Management Function (SMF) 214, a User Plane Function (UPF) 216, and a Charging Function (CHF) 218.

The interfaces N1 through N15 define communications and/or protocols between each NF as described in relevant standards. The UPF 216 is part of the user plane and the AMF 210, SMF 214, PCF 212, AUSF 206, and UDM 208 are part of the control plane. One or more UPFs can connect with one or more data networks (DNs) 220. The UPF 216 can be deployed separately from control plane functions. The NFs of the control plane are modularized such that they can be scaled independently. As shown, each NF service exposes its functionality in a Service Based Architecture (SBA) through a Service Based Interface (SBI) 221 that uses HTTP/2. The SBA can include a Network Exposure Function (NEF) 222, an NF Repository Function (NRF) 224, a Network Slice Selection Function (NSSF) 226, and other functions such as a Service Communication Proxy (SCP).

The SBA can provide a complete service mesh with service discovery, load balancing, encryption, authentication, and authorization for interservice communications. The SBA employs a centralized discovery framework that leverages the NRF 224, which maintains a record of available NF instances and supported services. The NRF 224 allows other NF instances to subscribe and be notified of registrations from NF instances of a given type. The NRF 224 supports service discovery by receipt of discovery requests from NF instances and, in response, details which NF instances support specific services.

The NSSF 226 enables network slicing, which is a capability of 5G to bring a high degree of deployment flexibility and efficient resource utilization when deploying diverse network services and applications. A logical end-to-end (E2E) network slice has pre-determined capabilities, traffic characteristics, and service-level agreements and includes the virtualized resources required to service the needs of a Mobile Virtual Network Operator (MVNO) or group of subscribers, including a dedicated UPF, SMF, and PCF. The wireless device 202 is associated with one or more network slices, which all use the same AMF. A Single Network Slice Selection Assistance Information (S-NSSAI) function operates to identify a network slice. Slice selection is triggered by the AMF, which receives a wireless device registration request. In response, the AMF retrieves permitted network slices from the UDM 208 and then requests an appropriate network slice of the NSSF 226.

The UDM 208 introduces a User Data Convergence (UDC) that separates a User Data Repository (UDR) for storing and managing subscriber information. As such, the UDM 208 can employ the UDC under 3GPP TS 22.101 to support a layered architecture that separates user data from application logic. The UDM 208 can include a stateful message store to hold information in local memory or can be stateless and store information externally in a database of the UDR. The stored data can include profile data for subscribers and/or other data that can be used for authentication purposes. Given a large number of wireless devices that can connect to a 5G network, the UDM 208 can contain voluminous amounts of data that is accessed for authentication. Thus, the UDM 208 is analogous to a Home Subscriber Server (HSS) and can provide authentication credentials while being employed by the AMF 210 and SMF 214 to retrieve subscriber data and context.

The PCF 212 can connect with one or more Application Functions (AFs) 228. The PCF 212 supports a unified policy framework within the 5G infrastructure for governing network behavior. The PCF 212 accesses the subscription information required to make policy decisions from the UDM 208 and then provides the appropriate policy rules to the control plane functions so that they can enforce them. The SCP (not shown) provides a highly distributed multi-access edge compute cloud environment and a single point of entry for a cluster of NFs once they have been successfully discovered by the NRF 224. This allows the SCP to become the delegated discovery point in a datacenter, offloading the NRF 224 from distributed service meshes that make up a network operator's infrastructure. Together with the NRF 224, the SCP forms the hierarchical 5G service mesh.

The AMF 210 receives requests and handles connection and mobility management while forwarding session management requirements over the N11 interface to the SMF 214. The AMF 210 determines that the SMF 214 is best suited to handle the connection request by querying the NRF 224. That interface and the N11 interface between the AMF 210 and the SMF 214 assigned by the NRF 224 use the SBI 221. During session establishment or modification, the SMF 214 also interacts with the PCF 212 over the N7 interface and the subscriber profile information stored within the UDM 208. Employing the SBI 221, the PCF 212 provides the foundation of the policy framework that, along with the more typical QoS and charging rules, includes network slice selection, which is regulated by the NSSF 226.

### A Personal Assistant to Aid in Trip Planning

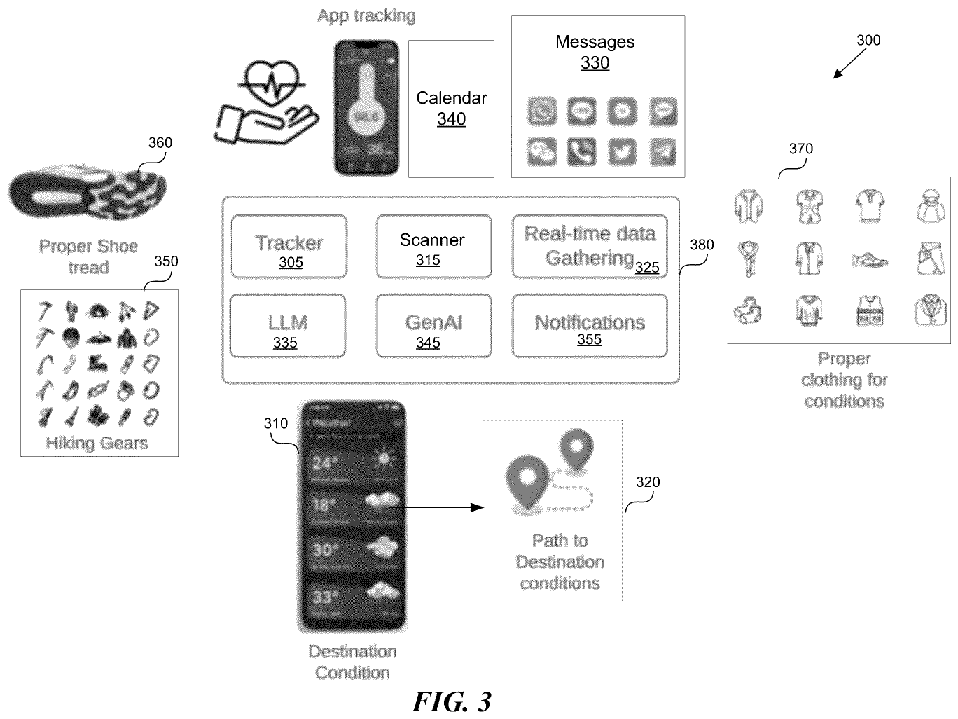

FIG. 3 is an overview of a personal digital assistant system 300 to aid in planning a trip. Most people today carry a UE 310 with them most of the time. Consequently, the UE 310 can serve as the primary resource to understand the user's contextual history and suggest activities 320, such as exercise routines or road trips. The UE 310 has access to all communication 330 directed to or from the user, facilitating better planning. The communication 330, e.g., messages, can include SMS and MMS messages, emails, voice messages, social media posts the user viewed or made, etc. The UE 310 can have access to or obtain the user's medical records, including the user's prescriptions. Consequently, the UE 310 can enable the preparation of necessary medical supplies prior to the activity 320, including filling prescriptions at home or the destination. Further, the UE 310 can safeguard against health-related risks.

The UE 310 can identify inappropriate gear and recommend suitable replacements, taking into account personal preferences and the nature of the trip. The UE 310 can assist in preparing required documents, such as passports, diving certificates, driver's licenses, etc. Based on when the trip is scheduled to start, the UE 310 can notify the user to initiate the application process for any missing documents.

Throughout the trip, the UE 310 can monitor conditions and provide timely alerts for any changes or considerations. For example, if the trip includes a kayaking activity, the UE 310 can monitor the tides and the weather forecast to detect changes to safety. If it becomes unsafe to be involved in the kayaking activity, the UE 310 can notify the user to cancel the activity.

This personal digital assistant system 300 can remain consistent and become even more adept over time thanks to the intelligent tracking and improvement capabilities of the generative AI over 5G high-speed Internet to deliver the information to users when they need it the most. The 5G high-speed telecommunication network can enable the user to receive videos from the destination, visually informing the user of the destination conditions.

The UE 310 can obtain and analyze messages 330 that are sent over the network 100. The messages 330 can include text messages, voice messages, emails, etc. The UE 310 can also obtain entries in a calendar 340 to obtain information about the trip or activity 320, start indicators, e.g., start date and time, end indicators, e.g., end date and time, location, and/or participants. The UE 310 can also notify the user to include individuals in a trip or an activity. The activity 320 can be a road trip, a short walk, a drive, or a flight out of town.

The UE 310 can obtain information about the user's health condition, prescriptions, and dietary preferences. This knowledge is crucial for trip planning, ensuring that necessary medications and nutritional needs are accounted for. If pre-filled prescriptions cannot be obtained locally during the trip, the UE 310 can assist in arranging pickups at the destination. The UE 310 can also alert the user of potential health hazards at the destination and can suggest the prescriptions and/or medicine to pack.

For certain activities 320, appropriate gear or equipment 350, 360 needs to be obtained for the activity, whether through purchases or utilizing existing items at home. If the user is in possession of the gear 350, 360, the UE 310 can scan the gear 350, 360 to check their condition, such as worn shoe treads or deteriorating car tire treads. If the UE 310 detects that the gear 350, 360 condition is not satisfactory, the UE 310 can prompt the user to address these issues before embarking on the journey by identifying the issue, such as “worn shoe treads” or “deteriorating tire treads.” For example, the UE 310 can obtain the weather conditions for the trip, such as a snowstorm in the mountains. The UE 310 can determine, based on the weather conditions, that the car tires need to be snow tires. Consequently, the UE 310 can scan the car tires to determine whether a different type of tire, such as the snow tire, is needed.

Further, the UE 310 can assist in proper preparation of clothing and belongings 370. If an item is missing from the appropriate gear 350, 360 or clothing and belongings 370, the UE 310 can prompt the user to purchase any missing items.

The subsystem 380 shows the enabling modules: tracker 305, scanner 315, real-time data gathering 325, large language model (LLM) 335, generative artificial intelligence (AI) 345, and notification module 355. The tracker 305 can gather information from the messages 330, the calendar 340, and other sources of information such as medical records. The scanner 315 can be a camera or a rangefinder configured to examine an item to determine whether the item's condition is satisfactory. Real-time data gathering 325 can continuously or periodically gather information about the destination, as described in this application. The LLM 335 can analyze the text obtained through the tracker 305, while the generative AI 345 can generate suggestions and/or notifications. The notification module 355 can present the suggestions and/or modifications to the user.

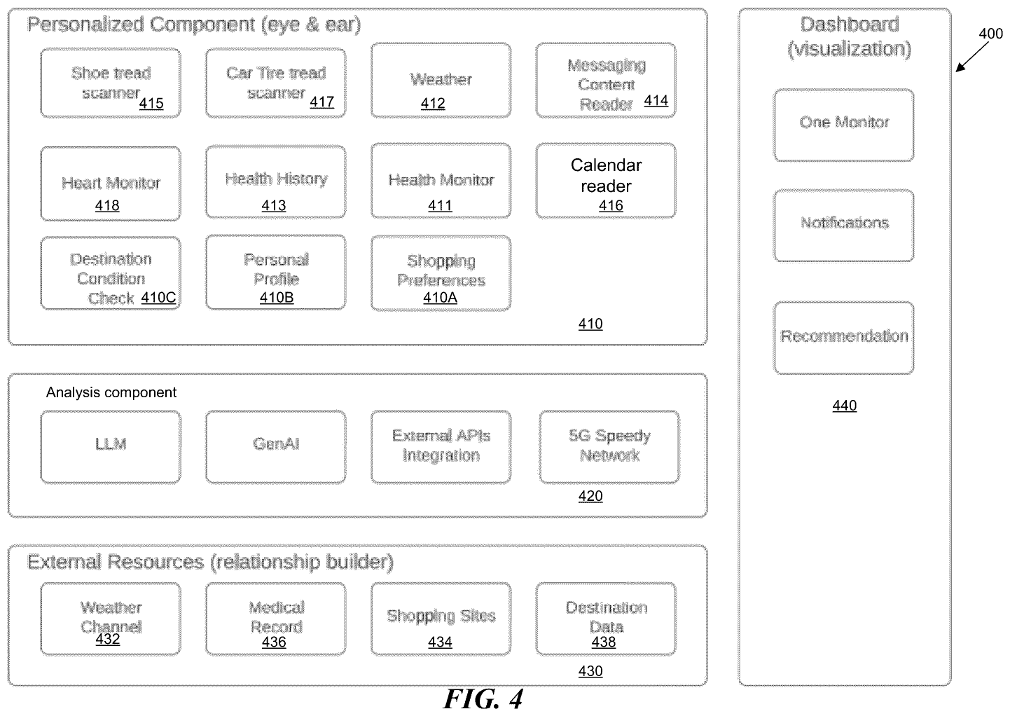

FIG. 4 shows the key components of a personal digital assistant system 400. The personal digital assistant system 400 can include the personalized component 410, the analysis component 420, the external resources component 430, and the dashboard/visualization component 440.

The personalized component 410 can incorporate a weather module 412 that obtains information about the weather at the destination. The personalized component 410 can incorporate messaging content reader 414 and the calendar reader 416. The messaging content reader 414 can obtain messages associated with the UE. The calendar reader 416 can obtain upcoming activities, e.g., trips, within the calendar. The personalized component 410 can incorporate heart monitor 418 that can be a device tracking the heart measurements, such as a smart watch, or a heart device, such as a pacemaker. The personalized component 410 can incorporate health monitor 411, which can include the heart monitor 418 and/or can include other modules tracking other health measurements such as oximeter measurements, gait measurements, etc. The personalized component 410 can incorporate health history 413 that can be a module that accesses the user's medical records for the user's permission and monitors prescription refills or doctor appointments. The personalized component 410 can incorporate scanners 415, 417 that can analyze shoe or tire treads to determine whether they are in a satisfactory condition for the activity. In addition, the personalized component 410 can include a shopping preferences module 410A tracking the user's purchase and rental history, personal profile module 410B storing user preferences, and a destination condition check 410C checking destination conditions including traffic, number of visitors, etc.

The external resources component 430 can integrate with various systems to collect external data, helping validate and construct the right scenarios for the user. For example, the external resources component 430 can obtain weather 432 at the destination, which influences how the user should dress upon arrival, contributing to a more enjoyable trip experience. The external resources component 430 can obtain shopping history, e.g., shopping sites 434 associated with the user, medical records 436 associated with the user, and destination data 438, such as activities information at the destination.

The analysis component 420 can utilize information from sensors and external resources to determine appropriate next steps for the activity. The analysis component 420 can recognize destination-specific requirements, incorporate personal style preferences, and recommend suitable equipment and accessories tailored to each user. As the user ages and destinations change in terms of laws, style, and behavior, the analysis component 420 can adapt accordingly.

The analysis component 420 can communicate its recommendations to the UE via the network 100 and through displays or alert notifications via a unified dashboard 440. The dashboard 440 can provide direct links to external sites for additional shopping or information, offering the user in-depth knowledge as needed. Over time, additional sensors and resources can be automatically added to the personal digital assistant system 400 based on the prior recommendations and user needs, eliminating the need for additional manual support to toggle their activation.

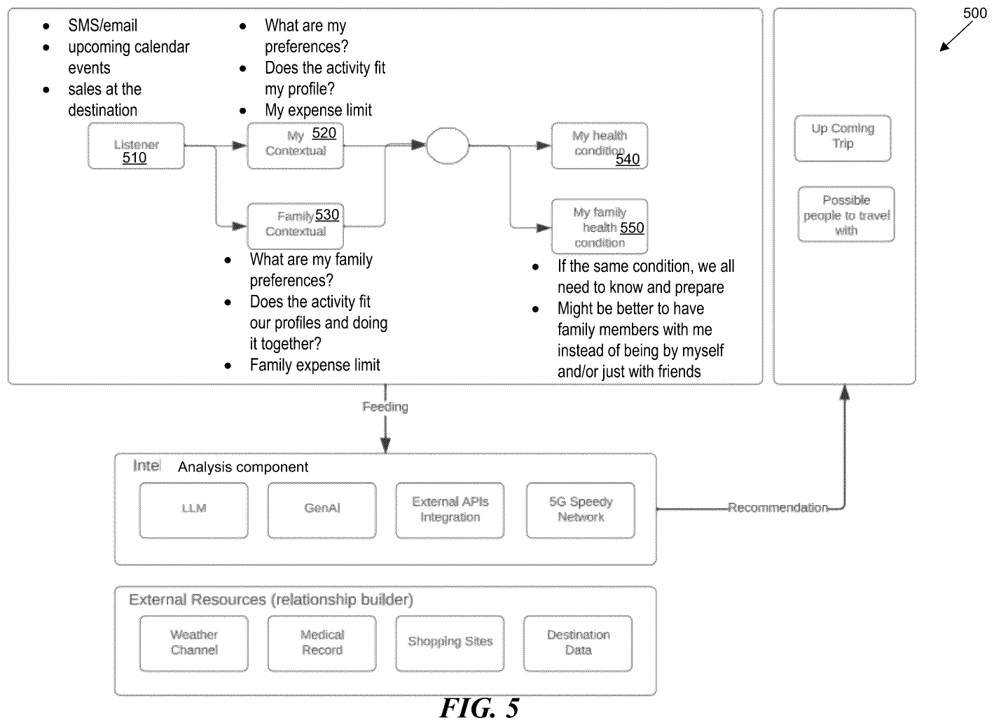

FIG. 5 shows a subsystem that recognizes a user's relationships and incorporates the relationships' preferences. The subsystem 500 can include a listener 510 that can obtain messages associated with the UE, calendar events, and information about the destination, such as sales at the destination. From the obtained information, the subsystem 500 can separate the information into user's profile 520 and user's family and friends profile 530. The user's profile 520 can include information about the user's preferences, such as preferred destinations, foods, expense limits, etc. Similarly, the family and friends profile 530 can include information about the family's preferences, including preferred destinations, foods, and expense limits. Further, from the obtained information, the subsystem 500 can separate information into user's health condition 540 and the family and friends health condition 550. The subsystem 500 can match the user with best travel companions based on similarity between the profiles 520, 530. Similarly, the subsystem 500 can determine whether the user needs assistance for a health condition and match the user with a friend or a family member who can assist with the health condition.

The subsystem 500 can also act as a personal accountant, assisting the user in staying within budget for a recommended trip. The subsystem 500 can explore group discounts to maximize the allocated budget.

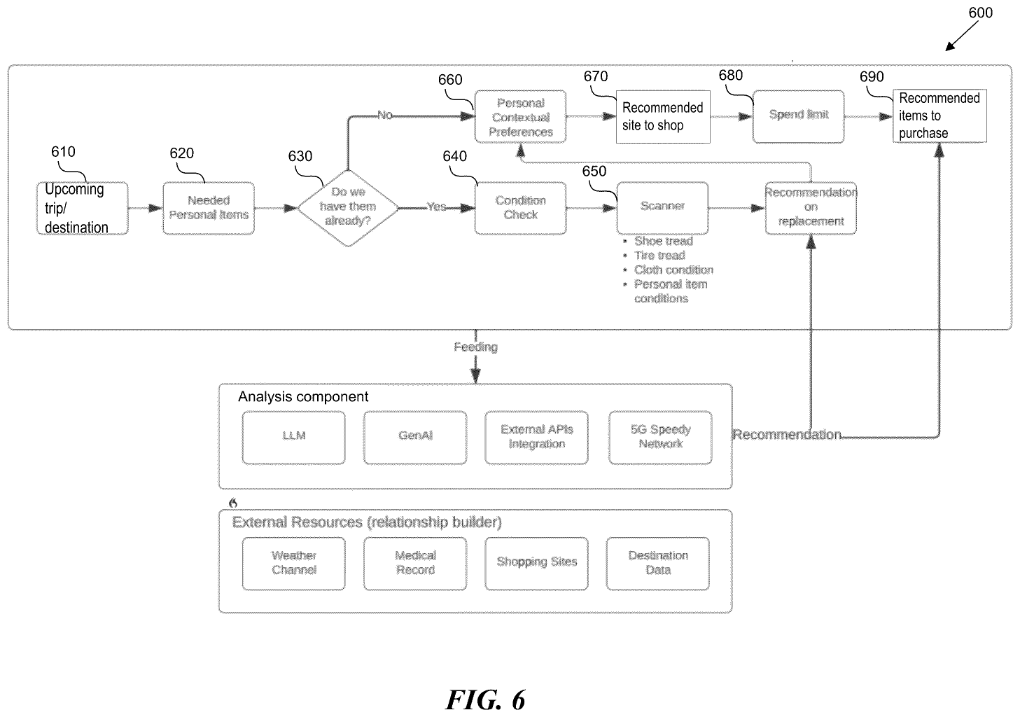

FIG. 6 presents a detailed flow of steps involved in recognizing the appropriate gear for an activity. When the system 600 identifies an upcoming activity and understands what the user needs to bring, the system can assist in inspecting the existing equipment to ensure its suitability. Equipped with a built-in scanner for examining shoe and tire treads, the system 600 can prevent potential safety issues during the activity.

This capability is crucial because anticipation of safety issues minimizes the risk for users who might otherwise be unaware of potential hazards. For instance, walking on a steep trail during wet weather conditions without proper shoe tread or driving through mountain passes with worn tires or without required chains poses risks to both the user and other drivers. The system 600 can also suggest additional gear based on common needs at the destination and specific needs related to the user's current health condition to support activities at the destination.

Avoiding over- or underdressing is a common concern for travelers, and having this information in advance can be invaluable. Understanding the typical dressing style at the destination becomes a key factor in contextualizing the user's preferences. If the user lacks any necessary items, the system 600 can recommend suitable shopping sites, taking into account upcoming discounts to fit within the user's budget.

In step 610, the system 600 can obtain an indication of the upcoming activity. In step 620, based on the indication of the upcoming activity, the system 600 can identify needed items. For example, if the activity is skiing, the system 600 can identify skis, ski clothes, ski boots, ski poles, and hand and toe warmers as needed equipment. In step 630, the system 600 can determine whether the user already has the needed equipment by obtaining the user's purchasing history, including rental history.

If the user's purchasing history includes the needed items, the system can perform a condition check in step 640 to determine whether the item is in satisfactory condition. To perform the condition check, in step 650, the system 600 can scan the item. For example, the system 600 can scan shoe treads, tire treads, cloth condition, or skis. If the system 600 determines that there is wear and tear, such that the treads are not deep enough, that there are tears in the clothes, or that there are gashes in the skis, the system 600 can suggest that the item be repaired or purchased. To make the suggestion, the system can consider the user's personal preferences in step 660.

If the user's purchasing history does not include the needed items, the system 600 can go to step 660. In step 660, the system 600 can consider the user's personal preferences, such as whether to repair or buy and/or preferred sites to shop. Based on the preferred shopping sites or repair sites, the system 600 can determine a cost of repair and/or cost of purchasing a new item in step 670. In step 680, the system 600 can obtain the spending limit, e.g., a budget, associated with the user. In step 690, based on the spending limit, the system can suggest a repair or a purchase and provide instructions on how to repair or purchase the item.

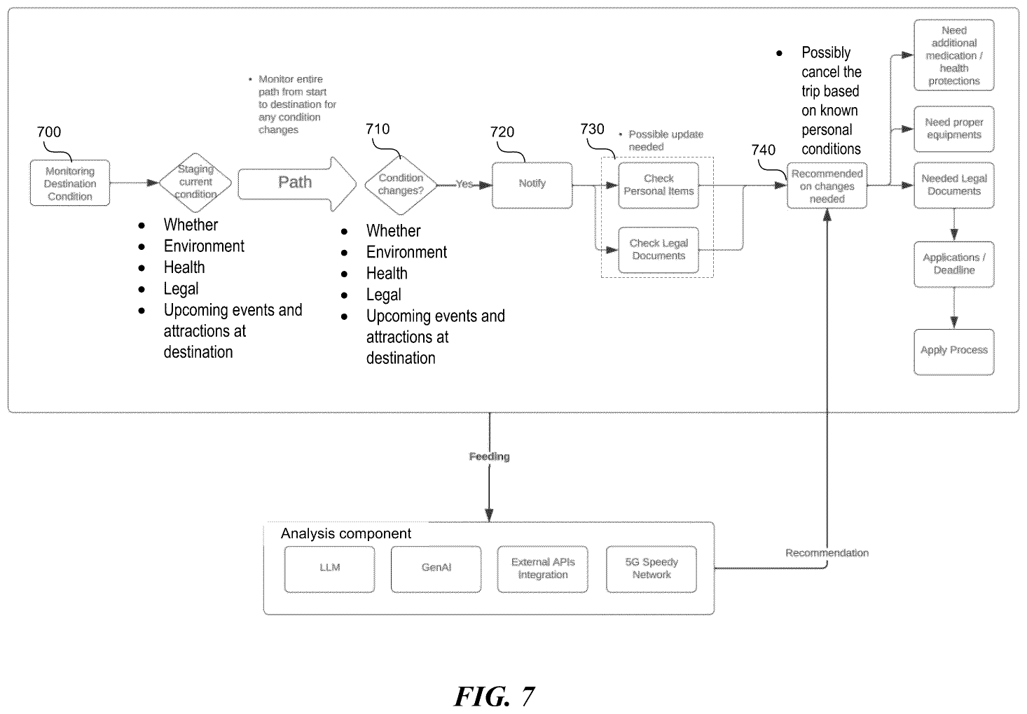

FIG. 7 illustrates monitoring of various aspects related to the destination and actions to take if changes occur. Every location comes with its unique customs and requirements, and every traveler aspires to be as informed as a local resident. The personal assistant described in this application can find out local requirements and regulations in the area, such as “no turn on red” regulations, which many travelers may overlook. Once found out, the personal assistant can determine which of the local requirements and regulations are different from the requirements and regulations at the user's own place and inform the user about the differences.

Considering the user's intent for the trip, activities scheduled by the user, and unscheduled activities available at the destination, the personal assistant can determine the appropriate clothing and gear. In addition, weather and environmental conditions may change at the destination, and the personal assistant can monitor these conditions prior to departure to facilitate proper preparation.

Over time, new events or activities may emerge at the destination that align with the user's interests. The personal assistant can monitor the new events or activities and suggest them to the user. If the user is interested, the personal assistant can suggest packing additional gear or buy admission tickets for the user and the user's travel group.

During a long road trip with multiple stops, conditions along the route may evolve. The personal assistant can monitor the road changes, adjust the route, and inform the user of any detected changes.

In step 700, the personal assistant can monitor destination conditions such as weather, environment, user's health, legal requirements, and activities such as events and attractions. For example, if the activity is a hike and the destination is a trailhead of the hike, the personal assistant can monitor the conditions along the length of the whole hike.

In step 710, the personal assistant can detect any changes in the destination conditions. In step 720, the personal assistant can notify the user of the changes.

In step 730, the personal assistant can check whether a change to the activity or items such as gear or legal papers brought to the activity are needed. In step 740, upon determining that the change to the activity is needed, the personal assistant can make a recommendation to the user. For example, the recommendation can indicate to cancel the trip, to bring additional medication, to bring additional equipment, to bring additional legal documents, etc. If additional legal documents are needed, the personal assistant can inform the user of when the deadline to obtain or file an application for the additional documents is and what the application process is.

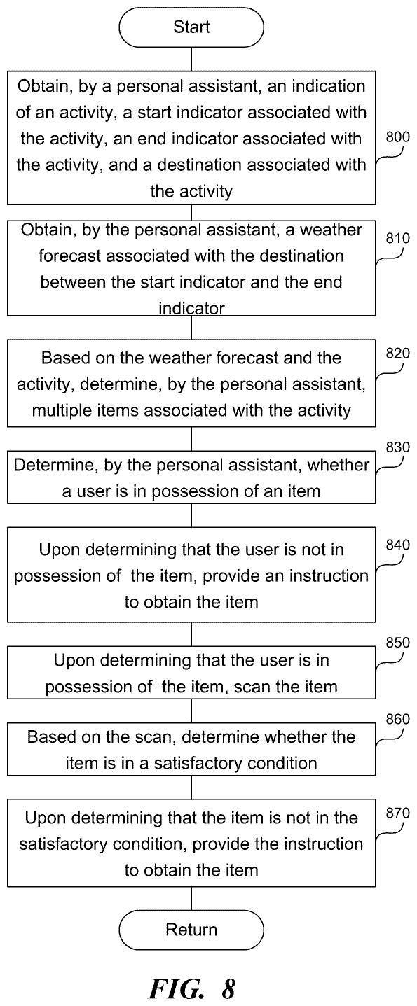

FIG. 8 is a flowchart of a method to aid in trip planning. A hardware or software processor executing instructions described in this application can implement a personal assistant associated with a UE operating on a 5G wireless telecommunication network, such as a 5G network. In step 800, the processor can obtain an indication of an activity, a start indicator associated with the activity, an end indicator associated with the activity, and a destination associated with the activity. The processor can obtain the information from a calendar or from emails and messages on the UE. The activity can be an upcoming trip or an upcoming activity entered into the calendar. The start indicator and the end indicator can be a start time or date and an end time or date, respectively.

In step 810, the processor can obtain, by the personal assistant, weather forecasts associated with the destination between the start indicator and the end indicator. In step 820, based on the weather forecast and the activity, the processor can determine, by the personal assistant, multiple items associated with the activity, such as clothing, legal documents, or equipment. Legal documents can include a permit, a passport, a license, a certificate, etc. The equipment can include hiking shoes, fishing equipment, skiing equipment, etc.

In step 830, the processor can determine whether a user is in possession of an item among the multiple items. In step 840, upon determining that the user is not in possession of the item among the multiple items, the processor can provide an instruction on how to obtain the item, such as by providing a link to purchase the item.

In step 850, upon determining that the user is in possession of the item among the multiple items, the processor can scan the item using a camera or a rangefinder. In step 860, based on the scan, the processor can determine whether the item is in a satisfactory condition. To determine whether the item is in the satisfactory condition, the processor can check the depth of the tread on the hiking boots or tires, the cloth condition for any tears, the condition of skis or snowboards for any gashes, etc.

In step 870, upon determining that the item is not in the satisfactory condition, the processor can provide the instruction to obtain the item, such as by providing a link to purchase or rent the item. In addition, the processor can create a packing list including all the items needed for the activity.

To determine whether the user is in possession of the item among the multiple items, the processor can obtain a second multiplicity of items from a purchasing history associated with the user and determine whether the item is among the second multiplicity of items. Upon determining that the item is among the second multiplicity of items, the processor can determine that the user is in possession of the item. Upon determining that the item is not among the second multiplicity of items, the processor can determine that the user is not in possession of the item.

Prior to initiation of the activity, the processor can periodically determine whether a change in the weather, a change in an environment associated with a destination, or a change in the activity occurred. For example, the processor can monitor the changes at increasing frequency as the commencement of the activity approaches. Initially, the processor can monitor the changes every week more than a month prior to the activity, every day a week before the activity, or every hour in the last 2 days before the activity. Upon determining that the change in the weather, the change in the environment, or the change in the activity occurred, the processor can determine a second item that can address the change in the weather, the change in the environment, or the change in the activity. For example, if the forecast changes to rain, the processor can determine that a rain jacket is needed. The processor can determine whether the second item is among the multiple items. Upon determining that the second item is not among the multiple items, the processor can determine whether the second item can be obtained prior to the initiation of the activity, for example, by purchasing or renting the second item. Upon determining that the second item can be obtained prior to the initiation of the activity, the processor can provide instructions to obtain the second item. Upon determining that the second item cannot be obtained prior to the initiation of the activity, the processor can suggest canceling the activity.

The processor can obtain an indication of a document needed to engage in the activity, such as a passport, license, e.g., a diving license, health checks, e.g., proof of vaccination, etc. The processor can determine whether the user is in possession of the document by searching files associated with the user. Upon determining that the user is not in possession of the documents, the processor can provide instructions to the user to obtain the document.

The processor can obtain a medicine prescription associated with the user and a date of last refill associated with the medicine prescription. Based on the start indicator associated with the activity, the end indicator associated with the activity, and the date of the last refill associated with the medicine prescription, the processor can determine whether another refill is needed during the activity. Upon determining that another refill is needed during the activity, the processor can notify the user. In addition, the processor can provide instructions on how to obtain the refill during the activity.

The processor can obtain messages received by the UE operating on the 5G wireless telecommunication network. The messages can include SMS messages, MMS messages, Internet messages, cellular messages, emails, etc. Based on the messages, the processor can determine activity preferences associated with the user. Prior to the start indicator associated with the activity, the processor can obtain a second activity associated with the destination, where the second activity is different from the activity and where the second activity is not part of the activity. For example, the activity can be a trip to Hawaii, and the second activity can be golfing. The processor can determine whether the second activity falls within the activity preferences associated with the user. For example, the processor can determine whether the user is a golfer. Upon determining that the second activity falls within the activity preferences associated with the user, the processor can suggest the second activity to the user.

The processor can mine the user's budget preferences. The processor can obtain purchasing history associated with the user. Based on the purchasing history, the processor can determine a budget associated with the item. Upon determining that the user is not in possession of the item among the multiple items, the processor can search the Internet for the item for purchase within the budget associated with the item. The processor can provide the instruction to obtain the item, such as a link to purchase the item.

### Computer System

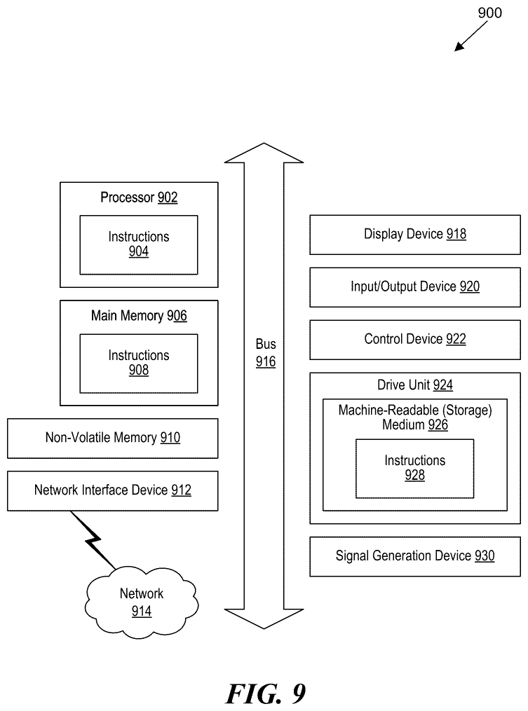

FIG. 9 is a block diagram that illustrates an example of a computer system 900 in which at least some operations described herein can be implemented. As shown, the computer system 900 can include: one or more processors 902, main memory 906, non-volatile memory 910, a network interface device 912, a video display device 918, an input/output device 920, a control device 922 (e.g., keyboard and pointing device), a drive unit 924 that includes a machine-readable (storage) medium 926, and a signal generation device 930 that are communicatively connected to a bus 916. The bus 916 represents one or more physical buses and/or point-to-point connections that are connected by appropriate bridges, adapters, or controllers. Various common components (e.g., cache memory) are omitted from FIG. 9 for brevity. Instead, the computer system 900 is intended to illustrate a hardware device on which components illustrated or described relative to the examples of the figures and any other components described in this specification can be implemented.

The computer system 900 can take any suitable physical form. For example, the computing system 900 can share a similar architecture as that of a server computer, personal computer (PC), tablet computer, mobile telephone, game console, music player, wearable electronic device, network-connected (“smart”) device (e.g., a television or home assistant device), AR/VR systems (e.g., head-mounted display), or any electronic device capable of executing a set of instructions that specify action(s) to be taken by the computing system 900. In some implementations, the computer system 900 can be an embedded computer system, a system-on-chip (SOC), a single-board computer system (SBC), or a distributed system such as a mesh of computer systems, or it can include one or more cloud components in one or more networks. Where appropriate, one or more computer systems 900 can perform operations in real time, in near real time, or in batch mode.

The network interface device 912 enables the computing system 900 to mediate data in a network 914 with an entity that is external to the computing system 900 through any communication protocol supported by the computing system 900 and the external entity. Examples of the network interface device 912 include a network adapter card, a wireless network interface card, a router, an access point, a wireless router, a switch, a multilayer switch, a protocol converter, a gateway, a bridge, a bridge router, a hub, a digital media receiver, and/or a repeater, as well as all wireless elements noted herein.

The memory (e.g., main memory 906, non-volatile memory 910, machine-readable medium 926) can be local, remote, or distributed. Although shown as a single medium, the machine-readable medium 926 can include multiple media (e.g., a centralized/distributed database and/or associated caches and servers) that store one or more sets of instructions 928. The machine-readable medium 926 can include any medium that is capable of storing, encoding, or carrying a set of instructions for execution by the computing system 900. The machine-readable medium 926 can be non-transitory or comprise a non-transitory device. In this context, a non-transitory storage medium can include a device that is tangible, meaning that the device has a concrete physical form, although the device can change its physical state. Thus, for example, non-transitory refers to a device remaining tangible despite this change in state.

Although implementations have been described in the context of fully functioning computing devices, the various examples are capable of being distributed as a program product in a variety of forms. Examples of machine-readable storage media, machine-readable media, or computer-readable media include recordable-type media such as volatile and non-volatile memory 910, removable flash memory, hard disk drives, optical disks, and transmission-type media such as digital and analog communication links.

In general, the routines executed to implement examples herein can be implemented as part of an operating system or a specific application, component, program, object, module, or sequence of instructions (collectively referred to as “computer programs”). The computer programs typically comprise one or more instructions (e.g., instructions 904, 908, 928) set at various times in various memory and storage devices in computing device(s). When read and executed by the processor 902, the instruction(s) cause the computing system 900 to perform operations to execute elements involving the various aspects of the disclosure.

### Remarks

The terms “example,” “embodiment,” and “implementation” are used interchangeably. For example, references to “one example” or “an example” in the disclosure can be, but not necessarily are, references to the same implementation; and such references mean at least one of the implementations. The appearances of the phrase “in one example” are not necessarily all referring to the same example, nor are separate or alternative examples mutually exclusive of other examples. A feature, structure, or characteristic described in connection with an example can be included in another example of the disclosure. Moreover, various features are described that can be exhibited by some examples and not by others. Similarly, various requirements are described that can be requirements for some examples but not for other examples.

The terminology used herein should be interpreted in its broadest reasonable manner, even though it is being used in conjunction with certain specific examples of the invention. The terms used in the disclosure generally have their ordinary meanings in the relevant technical art, within the context of the disclosure, and in the specific context where each term is used. A recital of alternative language or synonyms does not exclude the use of other synonyms. Special significance should not be placed upon whether or not a term is elaborated or discussed herein. The use of highlighting has no influence on the scope and meaning of a term. Further, it will be appreciated that the same thing can be said in more than one way.

Unless the context clearly requires otherwise, throughout the description and the claims, the words “comprise,” “comprising,” and the like are to be construed in an inclusive sense, as opposed to an exclusive or exhaustive sense—that is to say, in the sense of “including, but not limited to.” As used herein, the terms “connected,” “coupled,” and any variants thereof mean any connection or coupling, either direct or indirect, between two or more elements; the coupling or connection between the elements can be physical, logical, or a combination thereof. Additionally, the words “herein,” “above,” “below,” and words of similar import can refer to this application as a whole and not to any particular portions of this application. Where context permits, words in the above Detailed Description using the singular or plural number may also include the plural or singular number, respectively. The word “or” in reference to a list of two or more items covers all of the following interpretations of the word: any of the items in the list, all of the items in the list, and any combination of the items in the list. The term “module” refers broadly to software components, firmware components, and/or hardware components.

While specific examples of technology are described above for illustrative purposes, various equivalent modifications are possible within the scope of the invention, as those skilled in the relevant art will recognize. For example, while processes or blocks are presented in a given order, alternative implementations can perform routines having steps, or employ systems having blocks, in a different order, and some processes or blocks may be deleted, moved, added, subdivided, combined, and/or modified to provide alternative or sub-combinations. Each of these processes or blocks can be implemented in a variety of different ways. Also, while processes or blocks are at times shown as being performed in series, these processes or blocks can instead be performed or implemented in parallel, or can be performed at different times. Further, any specific numbers noted herein are only examples such that alternative implementations can employ differing values or ranges.

Details of the disclosed implementations can vary considerably in specific implementations while still being encompassed by the disclosed teachings. As noted above, particular terminology used when describing features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed herein, unless the above Detailed Description explicitly defines such terms. Accordingly, the actual scope of the invention encompasses not only the disclosed examples but also all equivalent ways of practicing or implementing the invention under the claims. Some alternative implementations can include additional elements to those implementations described above or include fewer elements.

Any patents and applications and other references noted above, and any that may be listed in accompanying filing papers, are incorporated herein by reference in their entireties, except for any subject matter disclaimers or disavowals, and except to the extent that the incorporated material is inconsistent with the express disclosure herein, in which case the language in this disclosure controls. Aspects of the invention can be modified to employ the systems, functions, and concepts of the various references described above to provide yet further implementations of the invention.

To reduce the number of claims, certain implementations are presented below in certain claim forms, but the applicant contemplates various aspects of an invention in other forms. For example, aspects of a claim can be recited in a means-plus-function form or in other forms, such as being embodied in a computer-readable medium. A claim intended to be interpreted as a means-plus-function claim will use the words “means for.” However, the use of the term “for” in any other context is not intended to invoke a similar interpretation. The applicant reserves the right to pursue such additional claim forms either in this application or in a continuing application.

I/We claim:

1. A non-transitory, computer-readable storage medium comprising instructions recorded thereon, wherein the instructions, when executed by at least one data processor of a system, cause the system to:
obtain, by a personal assistant associated with a mobile device operating on a 5G wireless telecommunication network, an indication of an upcoming trip, a start indicator associated with the upcoming trip, an end indicator associated with the upcoming trip, a destination associated with the upcoming trip, and an activity associated with the destination;
obtain, by the personal assistant, a weather forecast associated with the destination between the start indicator and the end indicator;
based on the weather forecast and the activity associated with the destination, determine, by the personal assistant, multiple items associated with the upcoming trip;
determine, by the personal assistant, whether a user is in possession of an item among the multiple items;
upon determining that the user is not in possession of the item among the multiple items, provide a link to purchase the item;
upon determining that the user is in possession of the item among the multiple items, scan the item;
based on the scan, determine whether the item is in a satisfactory condition; and
upon determining that the item is not in the satisfactory condition, provide the link to purchase the item.

2. The non-transitory, computer-readable storage medium of claim 1, comprising instructions to:
prior to initiation of the activity, periodically determine whether a change in weather, a change in an environment associated with a destination, or a change in the activity occurred;
upon determining that the change in the weather, the change in the environment, or the change in the activity occurred, determine a second item that can address the change in the weather, the change in the environment, or the change in the activity;
determine whether the second item is among the multiple items;
upon determining that the second item is not among the multiple items, determine whether the second item can be obtained prior to the initiation of the activity;
upon determining that the second item can be obtained prior to the initiation of the activity, provide instructions to obtain the second item; and
upon determining that the second item cannot be obtained prior to the initiation of the activity, suggest canceling the activity.

3. The non-transitory, computer-readable storage medium of claim 1, wherein the instructions to determine, by the personal assistant, whether the user is in possession of the item among the multiple items comprise instructions to:
obtain a second multiplicity of items from a purchasing history associated with the user;
determine whether the item is among the second multiplicity of items;
upon determining that the item is among the second multiplicity of items, determine that the user is in possession of the item; and
upon determining that the item is not among the second multiplicity of items, determine that the user is not in possession of the item.

4. The non-transitory, computer-readable storage medium of claim 1, comprising instructions to:
obtain, by the personal assistant associated with the mobile device operating on the 5G wireless telecommunication network, an indication of a document needed to engage in the upcoming trip;
determine whether the user is in possession of the document by searching files associated with the user; and
upon determining that the user is not in possession of the document, provide instructions to the user to obtain the document.

5. The non-transitory, computer-readable storage medium of claim 1, comprising instructions to:
obtain a medicine prescription associated with the user, and a date of last refill associated with the medicine prescription;
based on the start indicator associated with the upcoming trip, the end indicator associated with the upcoming trip, and the date of the last refill associated with the medicine prescription, determine whether another refill is needed during the upcoming trip; and
upon determining that another refill is needed during the upcoming trip, notify the user.

6. The non-transitory, computer-readable storage medium of claim 1, comprising instructions to:
obtain messages received by the mobile device operating on the 5G wireless telecommunication network;
based on the messages, determine activity preferences associated with the user;
prior to the start indicator associated with the upcoming trip, obtain a second activity associated with the destination,
wherein the second activity is different from the activity, and
wherein the second activity is not part of the upcoming trip;
determine whether the second activity falls within the activity preferences associated with the user; and
upon determining that the second activity falls within the activity preferences associated with the user, suggest the second activity to the user.

7. The non-transitory, computer-readable storage medium of claim 1, comprising instructions to:
obtain purchasing history associated with the user;
based on the purchasing history, determine a budget associated with the item;
upon determining that the user is not in possession of the item among the multiple items, search the Internet for the item for purchase within the budget associated with the item; and
provide the link to purchase the item.

8. A method comprising:
obtaining, by a personal assistant associated with a UE operating on a wireless telecommunication network, an indication of an activity, a start indicator associated with the activity, an end indicator associated with the activity, and a destination associated with the activity;
obtaining, by the personal assistant, a weather forecast associated with the destination between the start indicator and the end indicator;
based on the weather forecast and the activity, determining, by the personal assistant, multiple items associated with the activity;
determining, by the personal assistant, whether a user is in possession of an item among the multiple items;
upon determining that the user is not in possession of the item among the multiple items, providing an instruction to obtain the item;
upon determining that the user is in possession of the item among the multiple items, scanning the item;
based on the scan, determining whether the item is in a satisfactory condition; and
upon determining that the item is not in the satisfactory condition, providing the instruction to obtain the item.

9. The method of claim 8, wherein determining, by the personal assistant, whether the user is in possession of the item among the multiple items comprises:
obtaining a second multiplicity of items from a purchasing history associated with the user;
determining whether the item is among the second multiplicity of items;
upon determining that the item is among the second multiplicity of items, determining that the user is in possession of the item; and
upon determining that the item is not among the second multiplicity of items, determining that the user is not in possession of the item.

10. The method of claim 8, comprising:
prior to initiation of the activity, periodically determining whether a change in weather, a change in an environment associated with a destination, or a change in the activity occurred;
upon determining that the change in the weather, the change in the environment, or the change in the activity occurred, determining a second item that can address the change in the weather, the change in the environment, or the change in the activity;
determining whether the second item is among the multiple items;
upon determining that the second item is not among the multiple items, determining whether the second item can be obtained prior to the initiation of the activity;
upon determining that the second item can be obtained prior to the initiation of the activity, providing instructions to obtain the second item; and
upon determining that the second item cannot be obtained prior to the initiation of the activity, suggesting canceling the activity.

11. The method of claim 8, comprising:
obtaining, by the personal assistant associated with the UE operating on the wireless telecommunication network, an indication of a document needed to engage in the activity;
determining whether the user is in possession of the document by searching files associated with the user; and
upon determining that the user is not in possession of the document, providing instructions to the user to obtain the document.

12. The method of claim 8, comprising:
obtaining a medicine prescription associated with the user, and a date of last refill associated with the medicine prescription;
based on the start indicator associated with the activity, the end indicator associated with the activity, and the date of the last refill associated with the medicine prescription, determining whether another refill is needed during the activity; and
upon determining that another refill is needed during the activity, notifying the user.

13. The method of claim 8, comprising:
obtaining messages received by the UE operating on the wireless telecommunication network;
based on the messages, determining activity preferences associated with the user;
prior to the start indicator associated with the activity, obtaining a second activity associated with the destination,
wherein the second activity is different from the activity, and
wherein the second activity is not part of the activity;
determining whether the second activity falls within the activity preferences associated with the user; and
upon determining that the second activity falls within the activity preferences associated with the user, suggesting the second activity to the user.

14. A system comprising:
at least one hardware processor; and
at least one non-transitory memory storing instructions, which, when executed by the at least one hardware processor, cause the system to:
obtain, by a personal assistant associated with a UE operating on a wireless telecommunication network, an indication of an activity, a start indicator associated with the activity, an end indicator associated with the activity, and a destination associated with the activity;
obtain, by the personal assistant, a weather forecast associated with the destination between the start indicator and the end indicator;
based on the weather forecast and the activity, determine, by the personal assistant, multiple items associated with the activity;
determine, by the personal assistant, whether a user is in possession of an item among the multiple items;
upon determining that the user is not in possession of the item among the multiple items, provide an instruction to obtain the item;
upon determining that the user is in possession of the item among the multiple items, scan the item;
based on the scan, determine whether the item is in a satisfactory condition; and
upon determining that the item is not in the satisfactory condition, provide the instruction to obtain the item.

15. The system of claim 14, wherein the instructions to determine, by the personal assistant, whether the user is in possession of the item among the multiple items comprise instructions to:
obtain a second multiplicity of items from a purchasing history associated with the user;
determine whether the item is among the second multiplicity of items;
upon determining that the item is among the second multiplicity of items, determine that the user is in possession of the item; and
upon determining that the item is not among the second multiplicity of items, determine that the user is not in possession of the item.

16. The system of claim 14, comprising instructions to:
prior to initiation of the activity, periodically determine whether a change in weather, a change in an environment associated with a destination, or a change in the activity occurred;
upon determining that the change in the weather, the change in the environment, or the change in the activity occurred, determine a second item that can address the change in the weather, the change in the environment, or the change in the activity;
determine whether the second item is among the multiple items;
upon determining that the second item is not among the multiple items, determine whether the second item can be obtained prior to the initiation of the activity;
upon determining that the second item can be obtained prior to the initiation of the activity, provide instructions to obtain the second item; and
upon determining that the second item cannot be obtained prior to the initiation of the activity, suggest to cancel the activity.

17. The system of claim 14, comprising instructions to:
obtain, by the personal assistant associated with the UE operating on the wireless telecommunication network, an indication of a document needed to engage in the activity;
determine whether the user is in possession of the document by searching files associated with the user; and
upon determining that the user is not in possession of the document, provide instructions to the user to obtain the document.

18. The system of claim 14, comprising instructions to:
obtain a medicine prescription associated with the user, and a date of last refill associated with the medicine prescription;
based on the start indicator associated with the activity, the end indicator associated with the activity, and the date of the last refill associated with the medicine prescription, determine whether another refill is needed during the activity; and
upon determining that another refill is needed during the activity, notify the user.

19. The system of claim 14, comprising instructions to:
obtain messages received by the UE operating on the wireless telecommunication network;
based on the messages, determine activity preferences associated with the user;
prior to the start indicator associated with the activity, obtain a second activity associated with the destination,
wherein the second activity is different from the activity, and
wherein the second activity is not part of the activity;
determine whether the second activity falls within the activity preferences associated with the user; and
upon determining that the second activity falls within the activity preferences associated with the user, suggest the second activity to the user.

20. The system of claim 14, comprising instructions to:
obtain purchasing history associated with the user;
based on the purchasing history, determine a budget associated with the item;
upon determining that the user is not in possession of the item among the multiple items, search the Internet for the item for purchase within the budget associated with the item; and
provide the instruction to obtain the item.
